(19) United States
(12) Patent Application Publication
(10) Pub. No.: US 2026/0014370 A1
(43) Pub. Date: Jan. 15, 2026

(54) ELECTRICAL STIMULATION CUFF LEADS AND SYSTEMS AND METHODS OF MAKING AND USING

(71) Applicant: Boston Scientific Neuromodulation Corporation, Valencia, CA (US)

(72) Inventors: Nathan Maas, Simi Valley, CA (US); Ranjan Krishna Mukhari Nageri, Valencia, CA (US); Chirag Shah, Valencia, CA (US); Adarsh Jayakumar, Valencia, CA (US); Sarvani Grandhe, Valencia, CA (US); Hari Hara Subramanian, Valencia, CA (US)

(21) Appl. No.: 19/261,781

(22) Filed: Jul. 7, 2025

Related U.S. Application Data
(60) Provisional application No. 63/668,928, filed on Jul. 9, 2024.

(51) Int. Cl. A61N 1/05

(52) U.S. Cl. CPC A61N 1/0558; A61N 1/0556

(57) ABSTRACT

A cuff lead can include securing rings disposed along the first and second edges of a cuff body and an elongate securing body for insertion through the interleaved securing rings to hold the securing rings of the first and second edge in an interleaved configuration. A cuff lead can include a slot portion disposed along a first edge of a cuff body, the slot portion defining an opening, and a tab disposed along a second edge of the cuff body for securing the cuff lead to a nerve. A cuff lead can include two cuff body pieces engaging each other to form a hinge for moving between an open position for receiving a nerve and closed position. A cuff lead can include a tunnel extending through a cuff body and an elongate body for insertion through the tunnel.

### CROSS-REFERENCE TO RELATED APPLICATIONS

This application claims the benefit under 35 U.S.C. § 119 (e) of U.S. Provisional Patent Application Ser. No. 63/668,928, filed Jul. 9, 2024, which is incorporated herein by reference.

### FIELD

The present disclosure is directed to the area of implantable electrical stimulation systems and methods of making and using the systems. The present disclosure is also directed to implantable electrical stimulation cuff devices, as well as methods of making and using the same.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is a cuff lead configured for electrical stimulation. The cuff lead includes a cuff having a cuff body with an exterior surface, an interior surface, a first edge, and a second edge opposite the first edge, a plurality of cuff electrodes disposed on the interior surface of the cuff body, and a plurality of securing rings disposed along the first and second edges of the cuff body, wherein the securing rings along the first edge are configured to interleave with the securing rings along the second edge when the first and second edges are brought together. The cuff lead further including an elongate securing body configured for insertion through the interleaved securing rings to hold the securing rings of the first and second edge in an interleaved configuration.

In at least some aspects, the cuff lead further includes a lead body coupled to the cuff and a plurality of first conductors extending through the lead body and the cuff with the first conductors electrically coupled to the cuff electrodes. In at least some aspects, the elongate securing body includes a securing pin configured for insertion through the interleaved securing rings. In at least some aspects, the cuff body is precurved prior to attaching the cuff lead to a nerve.

In at least some aspects, the elongate securing body includes a lead including a lead body and a plurality of lead electrodes disposed along a distal end portion of the lead body, wherein the lead body is configured for insertion through the interleaved securing rings. In at least some aspects, the cuff body further includes a plurality of contacts distributed among, and disposed on, the securing rings, wherein the contacts are electrically coupled to the cuff electrodes and the lead electrodes are configured to electrically couple to the contacts.

Another aspect is a method for attaching the cuff leads described above to a nerve. The method includes wrapping the cuff body around a portion of the nerve to interleave the securing rings of the first and second edges and inserting the elongate securing body through the interleaved securing rings.

In at least some aspects, the cuff body further includes a plurality of contacts distributed among, and disposed on, the securing rings, wherein the contacts are electrically coupled to the cuff electrodes, wherein the elongate securing body includes a lead including a lead body and a plurality of lead electrodes disposed along a distal end portion of the lead body, wherein the lead body is configured for insertion through the interleaved securing rings and the lead electrodes are configured to electrically couple to the contacts; wherein inserting the elongate securing body includes inserting the lead through the interleaved securing rings and aligning the contacts disposed on the securing rings with the lead electrodes of the lead.

In at least some aspects, the method further includes rotating the elongate securing body inserted through the interleaved securing rings to rotate a portion of the cuff body onto the securing rings to reduce a diameter of the cuff body around the nerve. In at least some aspects, rotating the elongate securing body includes rotating the elongate securing body using a tool that provides an indication of different levels of torque applied using the tool.

Yet another aspect is a cuff lead configured for electrical stimulation. The cuff lead includes a cuff having a cuff body with an exterior surface, an interior surface, a first edge, and a second edge opposite the first edge, a plurality of cuff electrodes disposed on the interior surface of the cuff body, a slot portion disposed along the first edge of the cuff body, the slot portion defining an opening, and a tab disposed along the second edge of the cuff body, wherein the tab and the slot portion are configured for insertion of the tab through the opening of the slot portion as the cuff body is disposed around a nerve. The cuff lead also includes a lead body coupled to the cuff and a plurality of first conductors extending through the lead body and the cuff with the first conductors electrically coupled to the cuff electrodes.

A further aspect is a method for attaching the preceding cuff lead to a nerve. The method includes wrapping the cuff body around a portion of the nerve; inserting the tab through the opening of the slot portion; and attaching the tab to the exterior surface of the cuff body.

Another aspect is a cuff lead configured for electrical stimulation. The cuff lead includes a cuff having a cuff body with two cuff body pieces, an exterior surface, and an interior surface, each of the cuff body pieces including an engagement portion and a tab, the engagement portions of the cuff body pieces engaging each other to form a hinge, wherein the cuff body is configured for operation using the tabs of the two cuff body pieces to move between an open position for receiving a portion of a nerve in the cuff body and a closed position to retain the received nerve in the cuff body, and a plurality of cuff electrodes disposed on the interior surface of the cuff body. The cuff lead further includes a lead body coupled to the cuff and a plurality of first conductors extending through the lead body and the cuff with the first conductors electrically coupled to the cuff electrodes.

In at least some aspects, the cuff body further includes a spring disposed in the hinge and configured to bias the cuff body to the closed position.

Yet another aspect is a method for attaching any one of the preceding cuff leads to a nerve. The method includes pushing the tabs of the two cuff body pieces toward each other to put the cuff body in the open position; when in the open position, receiving a portion of the nerve between the two cuff body pieces; and after receiving the portion of the nerve, moving the tabs of the two cuff body pieces away from each other to return the cuff body to the closed position.

A further aspect is a cuff lead configured for electrical stimulation. The cuff lead includes a cuff having a cuff body with an exterior surface, an interior surface, a first edge, and a second edge opposite the first edge, the cuff body defining a first opening on the exterior surface, a plurality of second openings along the first edge of the cuff body, a plurality of third openings along the second edge of the cuff body, and a tunnel extending through the cuff body and including a plurality of tunnel segments, wherein a first one of the tunnel segments extends from the first opening to a one of the second or third openings, the plurality of tunnel segments including a plurality of additional tunnel segments, each of the additional tunnel segments extending from a different one of the second openings to a different one of the third openings, and a plurality of cuff electrodes disposed on the interior surface of the cuff body. The cuff lead further includes an elongate body configured for insertion through all of the tunnel segments of the tunnel when the first and second edges of the cuff body are brought together with the tunnel segments forming a pathway through the cuff body.

In at least some aspects, the cuff body further includes a plurality of contacts distributed along the tunnel, wherein the contacts are electrically coupled to the cuff electrodes, wherein the elongate body includes a lead including a lead body and a plurality of lead electrodes disposed along the lead body, wherein the lead body is configured for at least partial insertion through the tunnel and the lead electrodes are configured to electrically couple to the contacts. In at least some aspects, the cuff lead further includes a lead body coupled to the cuff and a plurality of first conductors extending through the lead body and the cuff with the first conductors electrically coupled to the cuff electrodes.

Another embodiment is a method for attaching any one of the preceding the cuff leads to a nerve. The method includes wrapping the cuff body around a portion of the nerve; inserting the elongate body into and through the tunnel; and pulling or rotating the elongate body to decrease a diameter of the cuff body around the portion of the nerve.

In at least some aspects, the cuff body further includes a plurality of contacts distributed along the tunnel, wherein the contacts are electrically coupled to the cuff electrodes, wherein the elongate securing body includes a lead including a lead body and a plurality of lead electrodes disposed along a distal end portion of the lead body, the method further including aligning the lead electrodes with the contacts to electrically couple the lead electrodes to the contacts.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a cuff lead electrically coupled to a control module;

FIG. 2A is a schematic view of one embodiment of the control module of FIG. 1 configured and arranged to electrically couple to an elongated device;

FIG. 2B is a schematic view of one embodiment of a lead extension configured and arranged to electrically couple the elongated device of FIG. 2A to the control module of FIG. 1;

FIG. 3 is a schematic perspective view of one embodiment of a cuff of a cuff lead;

FIG. 4A is a schematic perspective view of a distal end portion of one embodiment of a cuff lead with a clip, where the clip is in an open position;

FIG. 4B is a schematic perspective view of the cuff lead of FIG. 4A with the clip in a closed position around a lead;

FIG. 5 is a schematic view of a distal end portion of a third embodiment of a cuff lead, flattened out for illustration, with a tab and slot arrangement;

FIG. 6A is a schematic view of a distal end portion of a fourth embodiment of a cuff lead, flattened out for illustration, with securing rings and a securing pin;

FIG. 6B is a schematic end view of the distal end portion of the cuff lead of FIG. 6A disposed around a nerve;

FIG. 6C is a schematic end view of the distal end portion of the cuff lead of FIG. 6A after rotating the securing pin around the nerve;

FIG. 7 is a schematic view of a distal end portion of a fifth embodiment of a cuff lead, flattened out for illustration, with securing rings and a lead, as well as a tool for rotating the lead;

FIG. 8 is a schematic perspective view of a distal end portion of a sixth embodiment of a cuff lead with a tunnel for an elongate body; and

FIG. 9 is a schematic block diagram of one embodiment of an electrical stimulation arrangement.

### DETAILED DESCRIPTION

The present invention is directed to the area of implantable electrical stimulation systems and methods of making and using the systems. The present invention is also directed to implantable electrical stimulation cuff devices, as well as methods of making and using the same.

Suitable implantable electrical stimulation systems include, but are not limited to, a least one lead with one or more electrodes disposed along a distal end portion of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,203,548; 7,244,150; 7,450,997; 7,596,414; 7,610,103; 7,672,734; 7,761,165; 7,783,359; 7,792,590; 7,809,446; 7,949,395; 7,974,706; 6,175,710; 6,224,450; 6,271,094; 6,295,944; 6,364,278; 6,391,985; 7,596,414; 7,974,706; 8,831,742; 8,423,157; 10,485,969; 10,493,269; 10,709,888 and 10,485,969; and U.S. Patent Application Publication Nos. 2007/0150036; 2008/0304830; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2017/0333692; 2018/0154156; 2022/0370793; and 2022/0395690, all of which are incorporated by reference in their entireties.

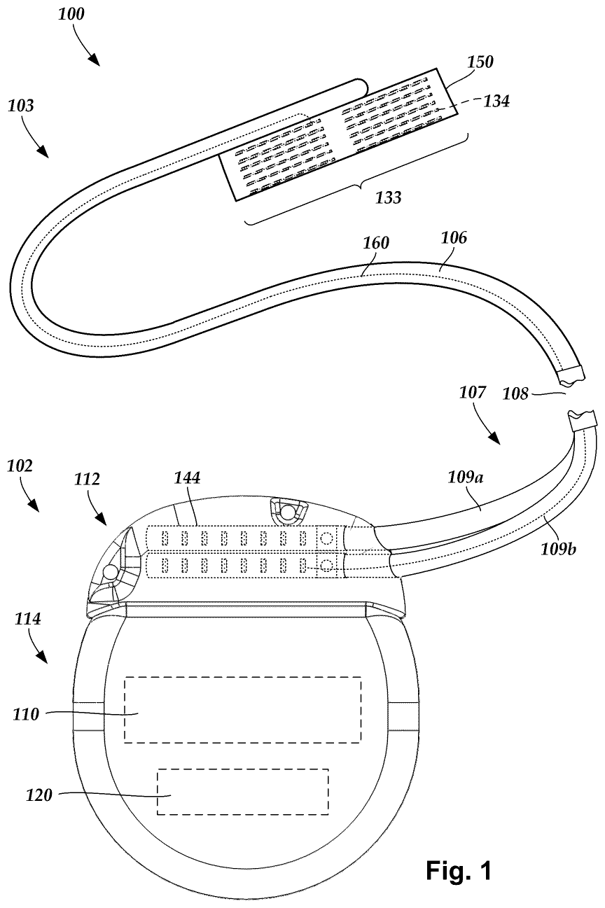

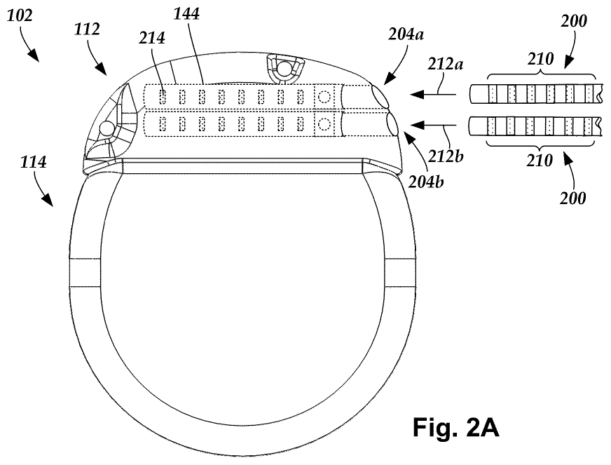

FIG. 1 illustrates schematically one embodiment of an electrical stimulation system 100. The electrical stimulation system includes a control module (e.g., a stimulator or pulse generator) 102 and a cuff lead 103 coupleable to the control module 102. The cuff lead 103 includes a cuff 150, and one or more lead bodies 106 attached to the cuff. The cuff lead 103 includes an array 133 of electrodes, such as electrode 134, disposed on the cuff 150 and an array of terminals (e.g., 210 in FIG. 2A-2B) attached to the one or more lead bodies 106. In at least some embodiments, the lead is isodiametric along at least a portion of the longitudinal length of the lead body 106. FIG. 1 illustrates one cuff lead 103 coupled to a control module 102. Other embodiments may include two, three, four, or more cuff leads 103 coupled to the control module 102. In yet other embodiments, a cuff lead 103 may be coupled to multiple control modules 102. For example, a cuff lead with 64 electrodes may be coupled to two control modules 102 that are capable of handling 32 electrodes each.

The cuff lead 103 can be coupled to the control module 102 in any suitable manner. In at least some embodiments, the cuff lead 103 couples directly to the control module 102. In at least some other embodiments, the cuff lead 103 couples to the control module 102 via one or more intermediate devices (200 in FIGS. 2A-2B). For example, in at least some embodiments one or more lead extensions 224 (see e.g., FIG. 2B) can be disposed between the cuff lead 103 and the control module 102 to extend the distance between the cuff lead 103 and the control module 102. Other intermediate devices may be used in addition to, or in lieu of, one or more lead extensions including, for example, a splitter, an adaptor, a device with multiplexing capability (as described, for example, in U.S. Pat. No. 10,350,413, which is incorporated by reference in its entirety) or the like or combinations thereof. It will be understood that, in the case where the electrical stimulation system 100 includes multiple elongated devices disposed between the cuff lead 103 and the control module 102, the intermediate devices may be configured into any suitable arrangement.

In FIG. 1, the electrical stimulation system 100 is shown having a splitter 107 configured and arranged for facilitating coupling of the cuff lead 103 to the control module 102. The splitter 107 includes a splitter connector 108 configured to couple to a proximal end of the cuff lead 103, and one or more proximal tails 109a and 109b configured and arranged to couple to the control module 102 (or another splitter, a lead extension, an adaptor, or the like). The splitter 107 and splitter connector 108 may be part of the cuff lead 103 or may be a separate component that attaches to the cuff lead.

The control module 102 typically includes a connector housing 112 and a sealed electronics housing 114. Stimulation circuitry 110 and an optional power source 120 are disposed in the electronics housing 114. A control module connector 144 is disposed in the connector housing 112. The control module connector 144 is configured and arranged to make an electrical connection between the cuff lead 103 and the stimulation circuitry 110 of the control module 102.

The electrical stimulation system or components of the electrical stimulation system, including the cuff lead 103 and the control module 102, are implanted into the body of a patient. The electrical stimulation system can be used for a variety of applications including, but not limited to, brain stimulation, neural stimulation, spinal cord stimulation, muscle stimulation, and the like.

The lead body 106 and cuff body 150 can be made of, for example, a non-conductive, biocompatible material such as, for example, silicone, polyurethane, polyetheretherketone (“PEEK”), epoxy, and the like or combinations thereof. The lead body 106 and cuff body 150 may be formed in the desired shape by any process including, for example, molding (including injection molding), casting, and the like. The non-conductive material typically extends from the distal end portion of the lead body 106 to the proximal end of the lead body 106.

Terminals (e.g., 210 in FIGS. 2A-2B) are typically disposed along the proximal end of the lead body 106 of the electrical stimulation system 100 (as well as any splitters, lead extensions, adaptors, or the like) for electrical connection to corresponding connector contacts (e.g., 214 and 240 in FIG. 2B). The connector contacts are disposed in connectors (e.g., 144 in FIGS. 1-2B; and 222 in FIG. 2B) which, in turn, are disposed on, for example, the control module 102 (or a lead extension, a splitter, an adaptor, or the like). Electrically conductive wires 160, cables, or the like (only one of which is shown in FIG. 1) extend from the terminals to the electrodes 134. Typically, one or more electrodes 134 are electrically coupled to each terminal. In at least some embodiments, each terminal is only connected to one electrode 134.

The electrically conductive wires (“conductors”) 160 (only one of which is illustrated in FIG. 1 for clarity) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end portion of the lead body 106, for example, for infusion of drugs or medication into the site of implantation of the lead body 106. In at least one embodiment, the one or more lumens are flushed continually, or on a regular basis, with saline, epidural fluid, or the like. In at least some embodiments, the one or more lumens are permanently or removably sealable at the distal end.

The conductors 160 (only one of which is illustrated in FIG. 1 for clarity) extend into the cuff 150 such that each conductor passes to provide a direct electrical connection with one of the electrodes 134 (e.g., one conductor is electrically connected with one electrode and so on). The lead body 106 may be attached to the cuff 150 using an suitable method or arrangement such as, but not limited to, molding or adhering the lead body 106 to the cuff 150. In other embodiments, the conductors 160 from within the lead body 106 are electrically coupled to the electrodes 134 using jumper, intermediate or transition wires from the lead body 106 to the electrodes 134.

Examples of cuff leads 103 and electrode arrangements for cuff leads can be found at U.S. Pat. Nos. 7,596,414; 7,974,706; 8,423,157; 10,485,969; 10,493,269; 10,709,888; and 10,814,127; U.S. Patent Application Publications Nos. 2017/0333692; 2018/0154156; 2022/0370793; and 2022/0395690; and U.S. Provisional Patent Application Ser. Nos. 63/539,774 and 63/549,797, all of which are incorporated herein by reference in their entireties.

FIG. 2A is a schematic side view of one embodiment of a proximal end of one or more elongated devices 200 configured and arranged for coupling to one embodiment of the control module connector 144. The one or more elongated devices may include, for example, the lead body 106, one or more intermediate devices (e.g., the lead extension 224 of FIG. 2B, an adaptor, or the like or combinations thereof), or a combination thereof. FIG. 2A illustrates two elongated devices 200 coupled to the control module 102. These two elongated devices 200 can be two tails as illustrated in FIG. 1 or two different leads or any other combination of elongated devices.

The control module connector 144 defines at least one port into which a proximal end of the elongated device 200 can be inserted, as shown by directional arrow 212. In FIG. 2A (and in other figures), the connector housing 112 is shown having two ports 204a and 204b. The connector housing 112 can define any suitable number of ports including, for example, one, two, three, four, five, six, seven, eight, or more ports.

The control module connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIG. 1) disposed at a distal end portion of the cuff lead 103. Examples of connectors in control modules are found in, for example, U.S. Pat. Nos. 7,244,150 and 8,224,450, which are incorporated by reference in their entireties.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 2B, the lead extension connector 222 is shown disposed at a distal end portion 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the cuff lead 103 (or other elongated device 200). The lead extension 224 may include a plurality of electrically conductive wires (not shown) that electrically couple the connector contacts 240 to a proximal end 248 of the lead extension 224 that is opposite to the distal end portion 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

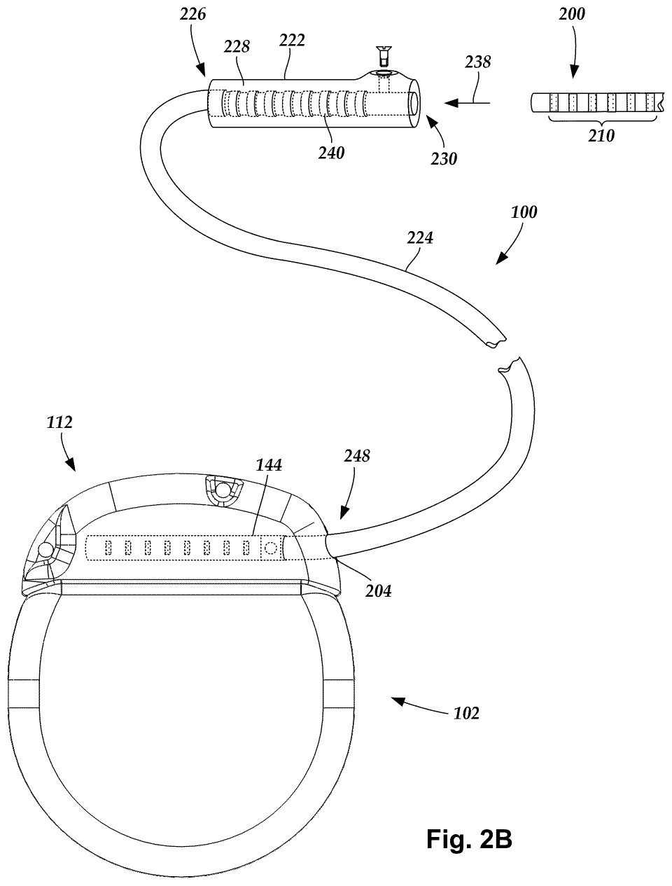

The arrangement of electrodes of a cuff can be used for selectivity of the portions of the nerve that are stimulated. FIG. 3 illustrates one embodiment of a cuff 350 of a cuff lead 103 (FIG. 1). The cuff 350 includes a cuff body 352 with electrodes 334 disposed on an interior surface 354 of the cuff body and arranged around the circumference of the cuff body in two sets 356a, 356b. In the illustrated embodiment, each set 356a, 356b includes sixteen longitudinal electrodes 334. Any other suitable number of electrodes can be used including, but not limited to, 2, 4, 6, 8, 10, 12, 16, 20, 25, 28, 32, 36, 40, 48, 50, 64, 80, 100, 120, 128, 150, 200, 250, 256, or more electrodes. A cuff lead can have one, two, three, four, or more sets of electrodes 334. The number of electrodes 334 in a set can be the same for each set or can differ. In the illustrated embodiment, the electrodes 334 of each set are aligned longitudinally with electrodes of the other set. In other embodiments, the electrodes 334 of each set can be staggered or unaligned with the electrodes of the other set. Optionally, the cuff 350 includes one or more radial electrodes 358 that wrap around at least 75%, 80%, 90%, or 95% of the circumference of the cuff body 352. Any suitable arrangement of electrodes 334, 358 can be used in the cuff leads described herein. Examples of other arrangements of electrodes can be found in the references cited herein.

The electrodes 334, 358 can be formed using any conductive, biocompatible material. Examples of suitable materials include metals, alloys, conductive polymers, conductive carbon, and the like, as well as combinations thereof. In at least some embodiments, one or more of the electrodes 334 are formed from one or more of: platinum, platinum alloys such as platinum iridium, palladium alloys such as palladium rhodium, titanium, titanium alloys, nickel alloys, cobalt alloys, nickel/cobalt alloys, stainless steel, tantalum, conductive carbon, conductive plastics, epoxy or other adhesive filled with metallic powder, Nitinol™, or the like or any combination thereof. The electrodes 334, 358 can be formed by any suitable process including, but not limited to, machining, molding (for example, powdered metal molding), photolithography, additive techniques, stamping, or the like or any combination thereof. The cuff body 352 can be made of any suitable biocompatible, non-conductive materials including, for example, polymeric materials such as silicone or the like.

In at least some embodiments, the electrodes 334, 358 have a contact surface that is flush or slightly protruding (for example, no more than 200, 100, or 50 μm from the cuff body 352 which, at least in some circumstances, may reduce or eliminate physical pressure on the nerve. It will be recognized that the electrodes can be used to provide electrical stimulation or to sense electrical signals from tissue or any combination thereof.

In at least some embodiments, the interior surface 354 of the cuff body 352 can be coated with a cushioning layer to act as a cushion to reduce damage to the nerve. Examples of materials for the cushioning layer include, but are not limited to, paraffin, a combination of isotonic saline and artificial cerebrospinal fluid, or the like or any combination thereof. The cushioning layer is made of a material that permits flow of current from the electrodes 334 to the nerve through the cushioning layer.

To position the cuff 350 around the nerve, the cuff defines a slit 360 that extends the longitudinal length of the cuff body 352. In at least some embodiments, the nerve can be loaded into the interior 362 of the cuff body by opening the slit to fit the cuff body over the nerve.

Often conventional arrangements for stimulating peripheral nerves, such as the vagus nerve, include suturing leads to the nerve. These leads can be difficult to explant without inducing post-operative side effects, which may be permanent. There is a need for leads that can be attached to the nerve and can be more easily explanted. There is also a need for cuff leads that provide an ability to modify the diameter of the cuff body to provide a good fit of the cuff lead to the nerve without applying excessive pressure to the lead. Cuff leads are described herein that, at least in some embodiments, can meet one or more of these needs.

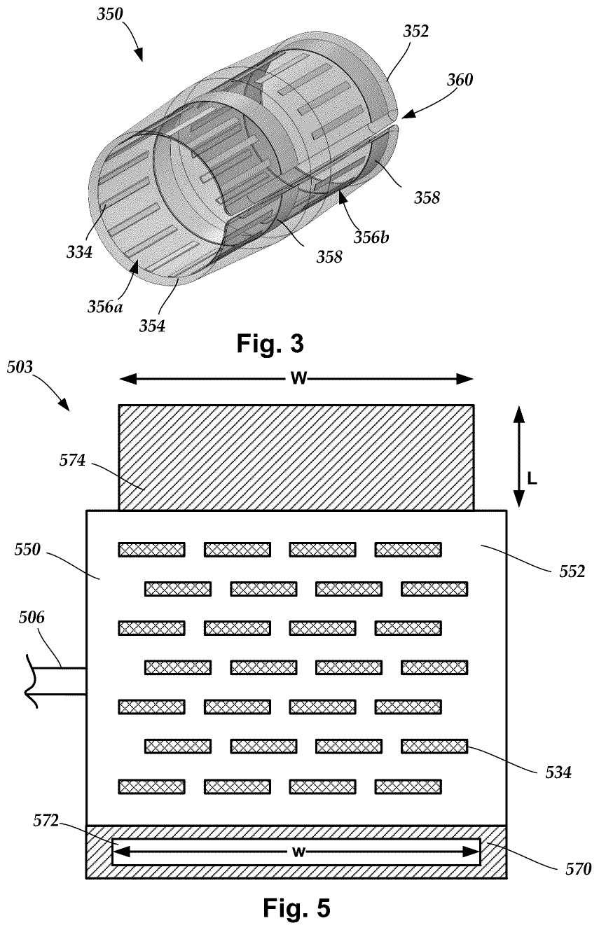

FIGS. 4A and 4B illustrate one embodiment of the distal end portion of a cuff lead 403, which can be the same as the cuff lead 103 of FIG. 1, or any other cuff lead described herein, except as described below. The cuff lead 403 includes a cuff 450 and a lead body 406. The cuff 450 includes a cuff body 452 with electrodes 434 disposed thereon. The cuff 450 defines a slit 460 and a clip mechanism 464 for opening the cuff (FIG. 4A-cuff 450 in open position) to load the nerve 451 into the cuff and then closing the cuff around the nerve (FIG. 4B-cuff 450 in closed position).

In this embodiment, the cuff body 452 includes two cuff body pieces 452a, 452b that each have an engagement portion 453 where the two cuff body pieces engage each other to form a hinge 465. In at least some embodiments, the hinge 465 includes a pin 455. The hinge 465 also includes a spring 466 (for example, a torsion spring) or other biasing element that biases the hinge 465 and two cuff body pieces 452a, 452b to the closed position (FIG. 4B). Each of the cuff body pieces 452a, 452b includes a tab 467 (or other hinge operation portion) adjacent to the engagement portion 453. A user pushes the tabs 467 of the two cuff body pieces 452a, 452b together to open the cuff body 452. The user releases the tabs 467 to return the cuff body to the closed position. In at least some embodiments, the user can use any suitable tool to push the tabs 467 together.

The components of the cuff 450, including the spring 466 and pin 455, are made of biocompatible materials, such as those described above for the electrodes and cuff body. For example, the spring 466 can be made of Nitinol or other biocompatible material with a high spring constant. In at least some embodiments, the stiffness of the spring 466 can be selected to facilitate opening of the cuff 450 by the user and still resist opening of the cuff 450 inadvertently when the cuff is implanted. In at least some embodiments, the cuff lead 403 can be explanted by pushing the tabs 467 together to open the cuff body 452 and then releasing the nerve 451 from the cuff 450.

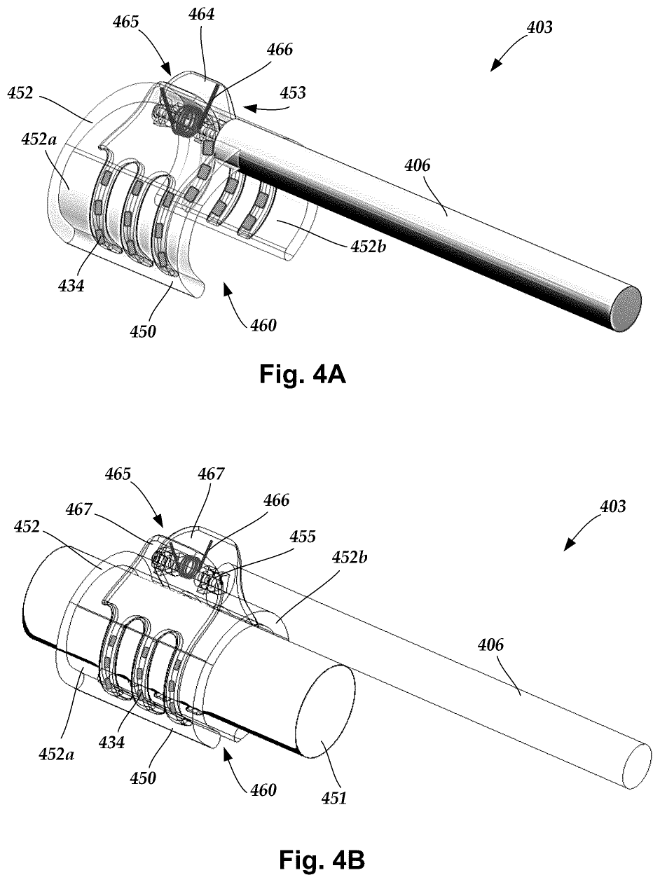

FIG. 5 illustrates another embodiment of a distal end portion of a cuff lead 503, which can be the same as the cuff lead 103 of FIG. 1, as well as any other cuff lead described herein, except as described below. The cuff lead 503 includes a cuff 550 and a lead body 506. The cuff 550 includes a cuff body 552 with electrodes 534 disposed thereon. The cuff body 552 also includes a tab 574 and a slot portion 570 defining an opening 572 (i.e., a slot).

In at least some embodiments, the cuff body 552 is formed as a flat mat or a mat with any suitable degree of bending or curving. In at least some embodiments, the cuff body 552 can be formed using any suitable technique including, but not limited to, molding, casting, forming as a sheet or a film, or the like or any combination thereof. In at least some embodiments, the cuff body 552 can be shaped in a curved or partially curved arrangement using, for example, heat, a scaffold or other external tool that the cuff body can be partially wrapped around, a mold, or the like or any combination thereof. In at least some embodiments, the electrodes 534 can be attached to the cuff body 552 using any suitable technique including, but not limited to, attaching with adhesive, molding (for example, insert molding) into the cuff body, using heat to adhere the electrodes to the cuff body, heating and pressing the electrodes into the cuff body, depositing and patterning electrode material on the cuff body, or the like or any combination thereof. It will be understood that the cuff of any other embodiment disclosed herein can be similarly made or arranged.

To attach the cuff 550 to a nerve, the cuff body 552 is wrapped around the nerve and the tab 574 is inserted through the opening 572 in the slot portion 570 and pulled a desired amount to fit or cinch the cuff body 552 around the nerve. In at least some embodiments, the amount of the tab 574 that passes through the opening 572 can be selected to provide a selected diameter of the cuff body 552. In at least some embodiments, the tab 574 can be pulled through the opening 572 until a selected degree of resistance to further pulling is met indicating a tightness of the fit of the cuff body 552 around the nerve.

After a desired amount of the tab 574 is pulled through the opening 572, the tab is then attached to the cuff body 552. Any suitable attachment mechanism or method can be used including, but not limited to, adhesive attachment, hook and loop attachment (for example, the tab 574 including a hook attachment or a loop attachment and an exterior surface of the cuff body 552 including the other one of the hook attachment or the loop attachment), button attachment, snap attachment, or the like or any combination thereof.

In at least some embodiments, a width, W, of the tab 574 is equal to or less than a width, w, of the opening 572 of the slot portion 570. In at least some embodiments, a length, L, of the tab 574 is selected to provide a range of diameters of the cuff body 552 around the nerve.

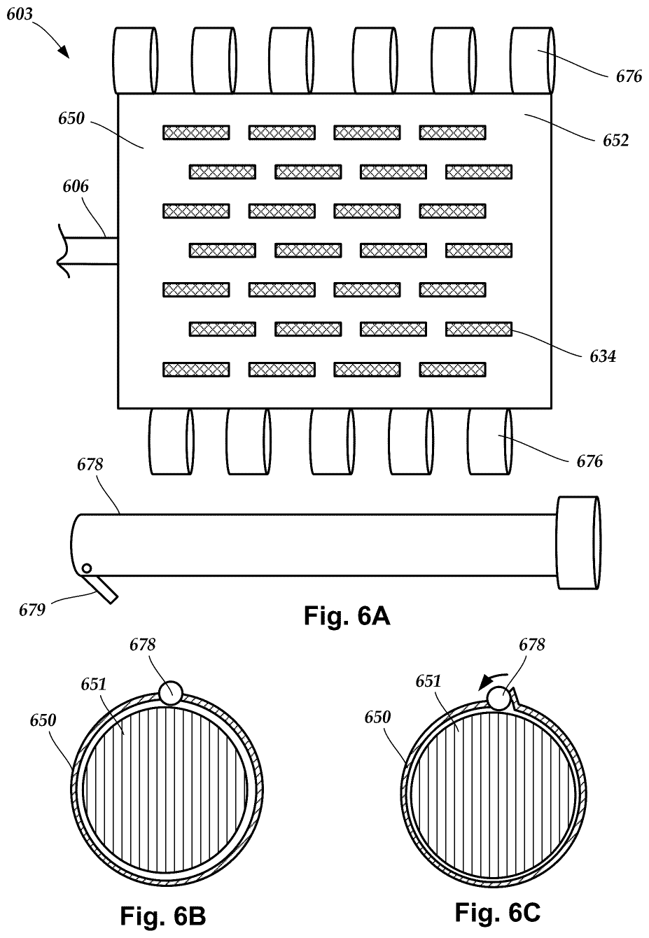

FIG. 6A illustrates yet another embodiment of a distal end portion of a cuff lead 603, which can be the same as the cuff leads 103, 503 of FIGS. 1 and 5, respectively, or any of the other cuff leads described herein, except as described below. The cuff lead 603 includes a cuff 650 and a lead body 606. The cuff 650 includes a cuff body 652 with electrodes 634 disposed thereon. The cuff body 652 also includes securing rings 676 disposed along two opposing sides of the cuff body, as well as an elongate securing body, such as securing pin 678. In at least some embodiments, the securing rings 676 are arranged to interleave as the securing rings disposed along the two opposing sides are brought together. Any other arrangement of the securing rings 676 can be used. The securing rings 676 can be a continuous loop or can have a starting end (for example, starting at one of the edges of the cuff body 652) and a terminal end (for example, terminating at the other one of the edges of the cuff body 652).

In at least some embodiments, the cuff body 652 is formed as a flat mat or a mat with any suitable degree of bending or curving. In at least some embodiments, the cuff body 652 can have surface roughness (e.g., pitting, deposition, or the like or any combination) to facilitate adhesion or input impedance. To attach the cuff 650 to a nerve, the cuff body 652 is wrapped around the nerve and the securing rings 676 disposed along the two opposing sides are brought together and aligned. The securing rings 676 can be attached to the cuff body 652 by any suitable method. The securing pin 678 is then inserted through the securing rings 676 to maintain the cuff 650 wrapped around the nerve. The securing rings 676 and securing pin 678 can be made of any suitable biocompatible material.

Some nerves are better stimulated when constricted and other nerves are better stimulated when not constricted. In at least some embodiments, the securing pin 678 can engage, or attach to, a portion of at least one of the securing rings 676 or the cuff body 652. For example, the securing pin 678 can include an extension 679 or other body that can engage, or attach to, at least one of the securing rings 676 or the cuff body 652 (or a combination thereof) when the securing pin 678 is inserted or when the securing pin 678 is rotated. FIG. 6B is a cross-sectional view of the cuff 650 disposed around a nerve 651. The securing pin 678 can be rotated to wrap a portion of the cuff body around the securing rings 676 and securing pin 678 to reduce the diameter of the cuff body 652, as illustrated in FIG. 6C. Such an arrangement can provide a desired tightness of the fit around the nerve. In at least some embodiments, adhesive, one or more sutures, any other locking mechanism, or the like or any combination thereof can be applied to the cuff body 652 or the securing pin 652 (or a combination thereof), after the desired diameter or tightness is obtained, to maintain the cuff body 652 in that configuration.

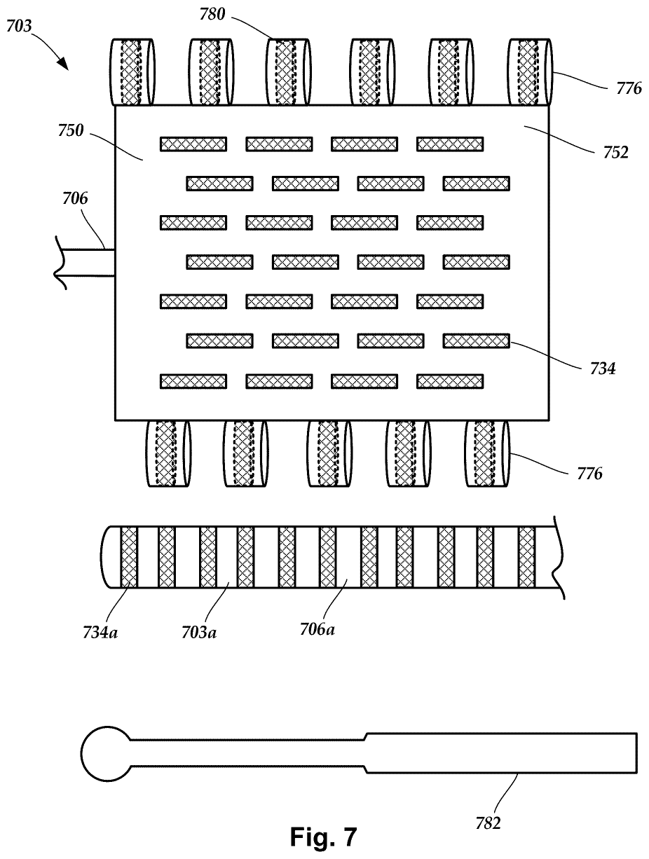

In at least some embodiments, the securing pin 676 can be rotated until a selected degree of resistance to further pulling is met indicating a tightness of the fit of the cuff body 652 around the nerve. In at least some embodiments, a tool (see, FIG. 7, tool 782), such as a torque wrench, can be used to rotate the securing pin 676 and apply a desired amount of torque to the securing pin. The torque is related to the tightness of the cuff body 652 around the nerve. In at least some embodiments, the tool provides an audible or haptic (or both) indication as the torque increases to particular values. In at least some embodiments, when a desired amount of rotation or torque has been achieved, the user can operate a control on, for example, the securing pin 676 or the cuff lead 603 to lock the rotation of the securing pin 676. In at least some embodiments, the user can operate a control on, for example, the securing pin 676 or the cuff lead 603 to release the lock. This can be useful for explant of the cuff lead 604 or for adjusting the fit of the cuff lead around the nerve.

FIG. 7 illustrates a further embodiment of a distal end portion of a cuff lead 703, which can be the same as the cuff leads 103, 503, 603 of FIGS. 1, 5, and 6, respectively, or any other cuff lead described herein, except as described below. The cuff lead 703 includes a cuff 750 and a lead body 706. The cuff 750 includes a cuff body 752 with electrodes 734 disposed thereon. The cuff body 752 also includes securing rings 776 disposed along two opposing sides of the cuff body and the cuff lead 703 also includes an elongate securing body in the form of a lead 703a that includes a lead body 706a and electrodes 734a disposed along the distal end portion of the lead 703a. Examples of leads, such as lead 703a, can be found in the references cited above. The lead 703a includes terminals (similar to terminals 210 of FIG. 2A) and conductors (similar to the conductors 160 of FIG. 1) electrically coupling the electrodes 734a to the terminals.

In at least some embodiments, the securing rings 776 are arranged to interleave as the securing rings disposed along the two opposing sides are brought together. Any other arrangement of the securing rings 776 can be used. The securing rings 776 can be a continuous loop or can have a starting end (for example, starting at one of the edges of the cuff body 752) and a terminal end (for example, terminating at the other one of the edges of the cuff body 752).

In at least some embodiments, the cuff body 752 is formed as a flat mat or a mat with any suitable degree of bending or curving. In at least some embodiments, the cuff body 652 can have surface roughness (e.g., pitting, deposition, or the like or any combination) to facilitate adhesion or input impedance. To attach the cuff 750 to a nerve, the cuff body 752 is wrapped around the nerve and the securing rings 776 disposed along the two opposing sides are brought together and aligned. The distal end portion of the lead 703a is then inserted through the securing rings 776 to maintain the cuff 750 wrapped around the nerve. The securing rings 676 can be made of any suitable biocompatible material.

In at least some embodiments, one or more of the securing rings 776 (such as, for example, every securing ring or only securing rings along one end portion of the cuff body 752) includes one or more contacts 780 disposed on an interior surface of the securing ring. The electrodes 734a of the lead 703a are arranged to make electrical contact with the contacts 780 of the securing rings 776 when the lead 703a is inserted into the securing rings. The contacts 780 of the securing rings 776 are electrically coupled to the electrodes 734 on the cuff body 752 using conductors (e.g., a portion of the conductor 160 of FIG. 1) and, accordingly, the electrode 734a are electrically coupled to the electrodes 734 on the cuff body 752 when the lead 703a is properly inserted into the securing rings 776.

In at least some embodiments, the lead 703a can engage, or attach to, a portion of at least one of the securing rings 776 or the cuff body 752. For example, the lead 703a can include an extension (for example, similar to extension 679 of FIG. 6) or other body that can engage, or attach to, at least one of the securing rings 776 or the cuff body 752 (or a combination thereof) when the lead 703a is rotated. The lead 703a can be rotated to wrap a portion of the cuff body around the securing rings 776 and lead 703a to reduce the diameter of the cuff body 752. Such an arrangement can provide a desired tightness of the fit around the nerve. In at least some embodiments, adhesive, one or more sutures, any other locking mechanism, or the like or any combination thereof can be applied to the cuff body 752 or lead 703a, after the desired diameter or tightness is obtained, to maintain the cuff body 752 in that configuration.

In at least some embodiments, the lead 703a can be rotated until a selected degree of resistance to further pulling is met indicating a tightness of the fit of the cuff body 752 around the nerve. In at least some embodiments, a tool 782, such as a torque wrench, can be used to rotate the lead 703a and apply a desired amount of torque to the lead. The torque is related to the tightness of the cuff body 752 around the nerve. In at least some embodiments, the tool 752 can be coupled to one of the proximal end portion or distal end portion of the lead 703a. In at least some embodiments, the tool provides an audible (e.g., a click) or haptic indication (or both types of indications) as the torque increases to particular values. In at least some embodiments, when a desired amount of rotation or torque has been achieved, the user can operate a control on, for example, the lead 703a or the cuff lead 703 to lock the rotation of the lead 703a. In at least some embodiments, the user can operate a control on, for example, the lead 703a or the cuff lead 703 to release the lock. This may be useful for explant of the cuff lead 703 or for adjusting the fit of the cuff lead around the nerve.

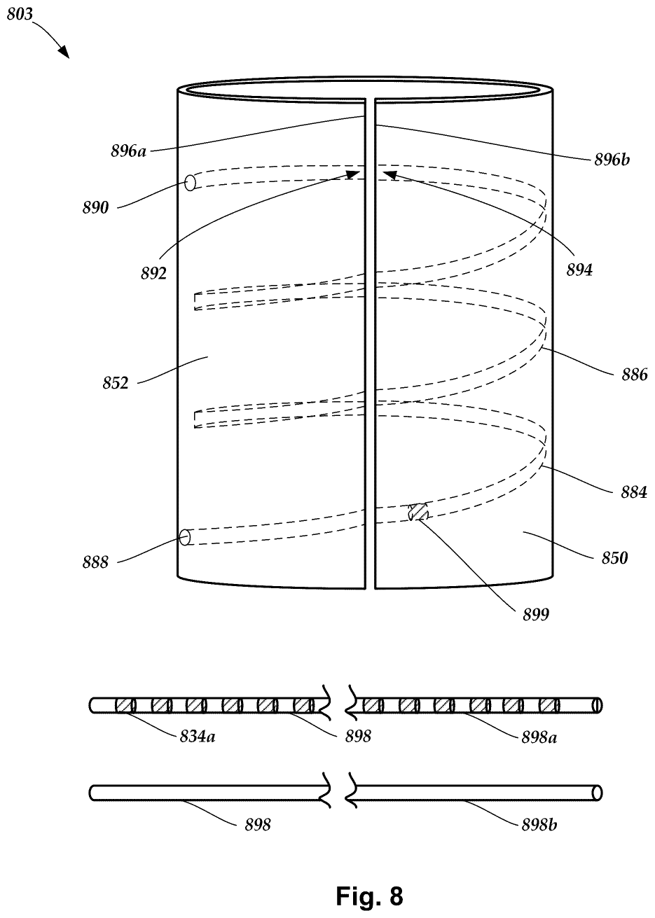

FIG. 8 illustrates yet another embodiment of a distal end portion of a cuff lead 803, which can be the same as the cuff leads 103, 503, 603, 703 of FIGS. 1, 5, 6, and 7, respectively, or any other cuff lead described herein, except as described below. The cuff lead 803 includes a cuff 850 and the cuff 850 includes a cuff body 852 with electrodes (not shown for clarity of illustration, but the same as the electrodes 134, 534, 634 in FIGS. 1, 5, and 6) disposed thereon.

The cuff body 852 defines a tunnel 884, with multiple tunnel segments 886, through the cuff body 852. The tunnel 884 originates at an opening 888 and, optionally, terminates at another opening 890. In at least some embodiments, the tunnel 884 is a helical or spiral tunnel. In at least some embodiments, the cuff body 852 also defines multiple second openings 892 along a first edge 896a of the cuff body and multiple third openings 894 along a second edge 896b of the cuff body 852. A first one of the tunnel segments 886 extends from the opening 888 to one of the second or third openings 892, 894. Other tunnel segments 886 extend from a different one of the second openings 892 to a different one of the third openings 894. In at least some embodiments, a last one of the tunnel segments 886 extends from one of the second or third openings 892, 894 to the opening 890.

The cuff lead 803 also includes an elongate body 898, such as, for example, a lead 898a (similar to the lead 703a, which has electrodes 734a) or a wire 898b or the like that does not include electrodes. After the cuff body 852 is placed around the nerve, the elongate body 898 is configured to be inserted through the opening 888 into the tunnel 884 and along the tunnel segments 886. In operation, the elongate body 898 is threaded through the tunnel 884 and either locked in the tunnel or extended out of the opening 890. In at least some embodiments, the distal end of the elongate body 898 can be attached to the cuff body 852, either within the tunnel 884 or on a surface of the cuff body, using adhesive, hook and loop fastener(s), button(s), snap(s), or any other suitable attachment mechanism or method or any combination thereof.

The elongate body 898 can be pulled or rotated to constrict the cuff body 852 around the nerve. In at least some embodiments, the elongate body 898 can be rotated using a tool, such as tool 782 (FIG. 7), to rotate the elongate body 898 and apply a desired amount of torque to the elongate body. The torque is related to the tightness of the cuff body 852 around the nerve. In at least some embodiments, the tool 752 can be coupled to one of (or either one of) the proximal end portion or distal end portion of the elongate body 898. In at least some embodiments, the tool provides an audible (e.g., a click) or haptic indication (or both types of indications) as the torque increases to particular values. In at least some embodiments, when a desired amount of rotation or torque has been achieved, the user can operate a control on, for example, the elongate body 898 or the cuff lead 803 to lock the rotation of the elongate body 898. In at least some embodiments, the user can operate a control on, for example, the elongate body 898 or the cuff lead 803 to release the lock.

In at least some embodiments, the elongate body 898 is a lead 898a with electrodes 834a. Contacts 899 (only one of which is illustrated for clarity) are distributed along the tunnel 884. The contacts 899 are electrically coupled to the electrodes (e.g., electrodes 734 of FIG. 7) of the cuff lead 850 using conductors. When the electrodes 834a of the lead 898a make electrical contact with the contacts 899, the electrodes of the lead are then electrically coupled to the electrodes (e.g., electrodes 734 of FIG. 7) of the cuff lead 850. When the elongate body 898 is not a lead (e.g., elongate body 898b), then the cuff lead 850 includes a lead body (such as the lead body 106, 406, 506, 606, 706 of FIG. 1, 4, 5, 6 or 7, respectively).

Any of the cuff leads (e.g., cuff leads 103, 403, 503, 603, 703, 803) can include at least one visible or radiopaque marker 840 disposed on the cuff body 852 or lead body (e.g., lead body 606 of FIG. 6) attached to the cuff body, or any combination thereof. In at least some embodiments, the marker(s) 840 can aid in determination of orientation of the cuff 850 and electrodes (e.g., electrodes 734 of FIG. 7) of the cuff and visualization of anatomical structures. In at least some embodiments, the marker(s) 840 can aid in orientation of the cuff body 852 relative to anatomical structures. In at least some embodiments, the cuff body 852 can be transparent so that marker(s) 840 and anatomical landmarks can be seen simultaneously.

In at least some embodiments, any of the cuffs described herein has an inner diameter in a range of 0.5 to 5.5 mm or in a range of 1 to 3 mm. In at least some embodiments, any of the cuffs described herein has a length of at least 5, 10, 20, 30, 40, or 50 mm or more. In at least some embodiments, any of the cuffs described herein is configured to fit around a portion of the vagus, splanchnic, hapatic, hypogastric, hypoglossal, sciatic, or other nerves.

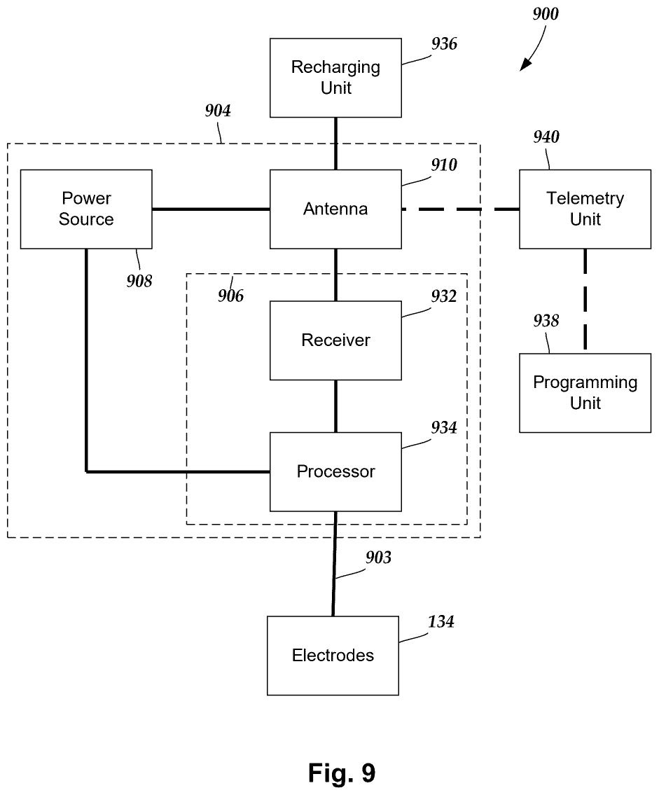

FIG. 9 is a schematic overview of one embodiment of components of an electrical stimulation system 900 with a cuff lead 903 and a stimulator 904 that includes stimulation circuitry 906, a power source 908, and an antenna 910. The electrical stimulation system can be, for example, an electrical stimulation system that includes any of the cuff leads described above. It will be understood that the electrical stimulation system can include more, fewer, or different components and can have a variety of different configurations including those configurations disclosed in the stimulator references cited herein.

If the power source 908 is a rechargeable battery or chargeable capacitor, the power source may be recharged/charged using the antenna 910, if desired. Power can be provided for recharging/charging by inductively coupling the power source 908 through the antenna 910 to a recharging unit 936 external to the user. Examples of such arrangements can be found in the references identified above.

In at least some embodiments, electrical current is emitted by the electrodes (such as electrodes 134 in FIG. 1) on the cuff lead 903 to stimulate nerve fibers, muscle fibers, or other body tissues near the electrical stimulation system. The stimulation circuitry 906 can include, among other components, a processor 934 and a receiver 932. The processor 934 is included to control the timing and electrical characteristics of the electrical stimulation system. For example, the processor 934 can, if desired, control one or more of the timing, frequency, strength, duration, and waveform of the pulses. In addition, the processor 934 can select which electrodes can be used to provide stimulation, if desired (see, for example, U.S. Pat. No. 8,412,345, which is incorporated herein by reference in its entirety). In some embodiments, the processor 934 selects which electrode(s) are cathodes and which electrode(s) are anodes. In some embodiments, the processor 934 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 938 that, for example, allows modification of pulse characteristics. In the illustrated embodiment, the processor 934 is coupled to a receiver 932 which, in turn, is coupled to the antenna 910. This allows the processor 934 to receive instructions from an external source to, for example, direct the pulse characteristics and the selection of electrodes, if desired.

In at least some embodiments, the antenna 910 is capable of receiving signals (e.g., RF signals) from an external telemetry unit 940 that is programmed by the programming unit 938. The programming unit 938 can be external to, or part of, the telemetry unit 940. The telemetry unit 940 can be a device that is worn on the skin of the user or can be carried by the user and can have a form similar to a pager, cellular phone, or remote control, if desired. As another alternative, the telemetry unit 940 may not be worn or carried by the user but may only be available at a home station or at a clinician's office. The programming unit 938 can be any unit that can provide information to the telemetry unit 940 for transmission to the stimulator 904. The programming unit 938 can be part of the telemetry unit 940 or can provide signals or information to the telemetry unit 940 via a wireless or wired connection. One example of a suitable programming unit is a computer operated by the user or clinician to send signals to the telemetry unit 940.

The signals sent to the processor 934 via the antenna 910 and the receiver 932 can be used to modify or otherwise direct the operation of the stimulation circuitry 906. For example, the signals may be used to modify the pulses of the electrical stimulation system such as modifying one or more of pulse duration, pulse frequency, pulse waveform, and pulse strength. The signals may also direct the stimulator 904 to cease operation, to start operation, to start charging the battery, or to stop charging the battery.

Optionally, the stimulator 904 may include a transmitter (not shown) coupled to the processor 934 and the antenna 910 for transmitting signals back to the telemetry unit 940 or another unit capable of receiving the signals. For example, the stimulator 904 may transmit signals indicating whether the stimulator 904 is operating properly or not or indicating when the battery needs to be charged or the level of charge remaining in the battery. The processor 934 may also be capable of transmitting information about the pulse characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected is:

1. A cuff lead configured for electrical stimulation, the cuff lead comprising:
a cuff comprising
a cuff body having an exterior surface, an interior surface, a first edge, and a second edge opposite the first edge,
a plurality of cuff electrodes disposed on the interior surface of the cuff body, and
a plurality of securing rings disposed along the first and second edges of the cuff body, wherein the securing rings along the first edge are configured to interleave with the securing rings along the second edge when the first and second edges are brought together; and
an elongate securing body configured for insertion through the interleaved securing rings to hold the securing rings of the first and second edge in an interleaved configuration.

2. The cuff lead of claim 1, further comprising a lead body coupled to the cuff and a plurality of first conductors extending through the lead body and the cuff with the first conductors electrically coupled to the cuff electrodes.

3. The cuff lead of claim 1, wherein the elongate securing body comprises a securing pin configured for insertion through the interleaved securing rings.

4. The cuff lead of claim 1, wherein the elongate securing body comprises a lead comprising a lead body and a plurality of lead electrodes disposed along a distal end portion of the lead body, wherein the lead body is configured for insertion through the interleaved securing rings.

5. The cuff lead of claim 4, wherein the cuff body further comprises a plurality of contacts distributed among, and disposed on, the securing rings, wherein the contacts are electrically coupled to the cuff electrodes and the lead electrodes are configured to electrically couple to the contacts.

6. The cuff lead of claim 1, wherein the cuff body is precurved prior to attaching the cuff lead to a nerve.

7. A method for attaching the cuff lead of claim 1 to a nerve, the method comprising;
wrapping the cuff body around a portion of the nerve to interleave the securing rings of the first and second edges; and
inserting the elongate securing body through the interleaved securing rings.

8. The method of claim 7, the cuff body further comprises a plurality of contacts distributed among, and disposed on, the securing rings, wherein the contacts are electrically coupled to the cuff electrodes, wherein the elongate securing body comprises a lead comprising a lead body and a plurality of lead electrodes disposed along a distal end portion of the lead body, wherein the lead body is configured for insertion through the interleaved securing rings and the lead electrodes are configured to electrically couple to the contacts;
wherein inserting the elongate securing body comprises inserting the lead through the interleaved securing rings and aligning the contacts disposed on the securing rings with the lead electrodes of the lead.

9. The method of claim 7, further comprising rotating the elongate securing body inserted through the interleaved securing rings to rotate a portion of the cuff body onto the securing rings to reduce a diameter of the cuff body around the nerve.

10. The method of claim 9, wherein rotating the elongate securing body comprises rotating the elongate securing body using a tool that provides an indication of different levels of torque applied using the tool.

11. A cuff lead configured for electrical stimulation, the cuff lead comprising:
a cuff comprising
a cuff body comprising two cuff body pieces, an exterior surface, and an interior surface, each of the cuff body pieces comprising an engagement portion and a tab, the engagement portions of the cuff body pieces engaging each other to form a hinge, wherein the cuff body is configured for operation using the tabs of the two cuff body pieces to move between an open position for receiving a portion of a nerve in the cuff body and a closed position to retain the received nerve in the cuff body, and
a plurality of cuff electrodes disposed on the interior surface of the cuff body;
a lead body coupled to the cuff; and
a plurality of first conductors extending through the lead body and the cuff with the first conductors electrically coupled to the cuff electrodes.

12. The cuff lead of claim 11, wherein the cuff body further comprises a spring disposed in the hinge and configured to bias the cuff body to the closed position.

13. The cuff lead of claim 11, wherein the lead body comprises a plurality of first conductors extending through the lead body and the cuff with the first conductors electrically coupled to the cuff electrodes.

14. A method for attaching the cuff lead of claim 11 to a nerve, the method comprising;
pushing the tabs of the two cuff body pieces toward each other to put the cuff body in the open position;
when in the open position, receiving a portion of the nerve between the two cuff body pieces; and
after receiving the portion of the nerve, moving the tabs of the two cuff body pieces away from each other to return the cuff body to the closed position.

15. A cuff lead configured for electrical stimulation, the cuff lead comprising:
a cuff comprising
a cuff body having an exterior surface, an interior surface, a first edge, and a second edge opposite the first edge, the cuff body defining a first opening on the exterior surface, a plurality of second openings along the first edge of the cuff body, a plurality of third openings along the second edge of the cuff body, and a tunnel extending through the cuff body and comprising a plurality of tunnel segments, wherein a first one of the tunnel segments extends from the first opening to a one of the second or third openings, the plurality of tunnel segments comprising a plurality of additional tunnel segments, each of the additional tunnel segments extending from a different one of the second openings to a different one of the third openings, and
a plurality of cuff electrodes disposed on the interior surface of the cuff body; and
an elongate body configured for insertion through all of the tunnel segments of the tunnel when the first and second edges of the cuff body are brought together with the tunnel segments forming a pathway through the cuff body.

16. The cuff lead of claim 15, wherein the cuff body further comprises a plurality of contacts distributed along the tunnel, wherein the contacts are electrically coupled to the cuff electrodes.

17. The cuff lead of claim 16, wherein the elongate body comprises a lead comprising a lead body and a plurality of lead electrodes disposed along the lead body, wherein the lead body is configured for at least partial insertion through the tunnel and the lead electrodes are configured to electrically couple to the contacts.

18. The cuff lead of claim 15, wherein the lead body comprises a plurality of first conductors extending through the lead body and the cuff with the first conductors electrically coupled to the cuff electrodes.

19. A method for attaching the cuff lead of claim 15 to a nerve, the method comprising:
wrapping the cuff body around a portion of the nerve;
inserting the elongate body into and through the tunnel; and
pulling or rotating the elongate body to decrease a diameter of the cuff body around the portion of the nerve.

20. The method of claim 19, wherein the cuff body further comprises a plurality of contacts distributed along the tunnel, wherein the contacts are electrically coupled to the cuff electrodes, wherein the elongate securing body comprises a lead comprising a lead body and a plurality of lead electrodes disposed along a distal end portion of the lead body,
the method further comprising aligning the lead electrodes with the contacts to electrically couple the lead electrodes to the contacts.
